(19) United States
(12) Patent Application Publication
(10) Pub. No.: US 2005/0288574 A1
(43) Pub. Date: Dec. 29, 2005

(54) Wireless (disposable) fiducial based registration and EM distoration based surface registration

(76) Inventors: Thomas M. Thornton, Harvard, MA (US); Peter T. Anderson, Andover, MA (US)

Correspondence Address: MCANDREWS HELD & MALLOY, LTD, 500 WEST MADISON STREET, SUITE 3400, CHICAGO, IL, 60661, US

(21) Appl. No.: 10/874,629

(22) Filed: Jun. 23, 2004

(51) Int. Cl. A61B 5/05

(52) U.S. Cl. 600/423

(57) ABSTRACT

The present invention relates to a method and system for performing automatic registration in medical imaging systems. One embodiment relates to a method of performing automatic registration comprising automatically locating at least one sensor having at least one known identifier. A coordinate is then determined for the at least one sensor, thereby performing automatic registration such that a visual model may be generated.

### BACKGROUND OF THE INVENTION

One or more embodiments relate to fiducial based registration. More specifically, embodiments relate to performing automatic registration (wireless and distortion based registration for example) in medical imaging systems.

Techniques for reconstructing models (3D visual models for example) from tomographic two-dimensional images are known. Some of these techniques include calibration techniques and use markers which act as references in space during image acquisition (using an X-ray, ultrasound or other imaging device or system for example). The positions of these markers in three-dimensional space are supposed to be known. The image acquisition geometry for each projection may be deduced using equations which are derived from the position of the markers on the projected images.

As provided previously, performing automatic registration in medical and surgical imaging, and in particular in intraoperative or perioperative imaging, one or more images are formed of at least one region of a patient's body (the cranium for example). A surgical tool or instrument may then be applied thereto, where the images may aid in the ongoing procedure. Such imaging may be used in surgical procedures including brain surgery and arthroscopic procedures on the knee, wrist, shoulder or spine, as well as certain types of angiography, cardiac procedures, interventional radiology and biopsies in which x-ray images may be taken to display, correct the position of, or otherwise navigate a tool or instrument involved in the procedure.

Several types of surgical procedures require very precise planning and control of the placement of elongated probes or other articles in tissue or bone where such placement is internal or difficult to view directly. In brain surgery for example stereotactic frames may be used to define the entry point, probe angle and probe depth to access a site in the brain, generally in conjunction with previously compiled three-dimensional diagnostic images (MRI, PET or CT scan images for example) which provide accurate tissue images. Such diagnostic systems have also been useful in the placement of pedicle screws in the spine, where visual and fluoroscopic imaging directions cannot capture an axial view necessary to center the profile of an insertion path in bone.

When used with existing image sets (CT, PET and MRI image sets for example), the previously recorded diagnostic image sets define a three dimensional rectilinear coordinate system by virtue of their precision scan formation or the spatial mathematics of their reconstruction algorithms. However, it may be necessary to correlate the available fluoroscopic views and anatomical features visible from the surface or in fluoroscopic images with features in the 3-D diagnostic images and with the external coordinates of the tools being employed. This is often accomplished by providing fiducials, including implanted fiducials and externally visible or trackable markers as provided previously, that may be imaged. A keyboard or mouse may be used as part of the known acquisition systems to identify the fiducials or markers in the various images. The fiducials or markers may then be used to identify common sets of coordinate registration points in the different images. Generally, such acquisition systems operate with an image display which is positioned in the surgeon's (or other user's) field of view, and which may display a few panels such as a selected MRI image and several x-ray or fluoroscopic views taken from different angles.

Correlation of patient anatomy or intraoperative images (fluoroscopic images for example) with precompiled 3-D diagnostic image data sets may also be complicated by intervening movement of the imaged structures, particularly soft tissue structures, between the times of original imaging and the intraoperative procedure. Thus, transformations between three or more coordinate systems for two sets of images and the physical coordinates in the operating room may require a large number of registration points to provide an effective correlation. For spinal tracking to position pedicle screws it may be necessary to initialize the tracking assembly on ten or more points on a single vertebra to achieve suitable accuracy. In cases where a growing tumor or evolving condition actually changes the tissue dimension or position between imaging sessions, further confounding factors may appear.

In theory, techniques using markers should provide better precision than techniques that don't use markers. In practice, it is often difficult to precisely determine the position of the markers in space. It is contemplated that, the markers may move slightly or even be missed during acquisition.

### BRIEF SUMMARY OF THE INVENTION

One or more embodiments relate to fiducial based registration. One or more embodiments relate to systems and methods for performing automatic registration using medical imaging systems or devices for example.

One embodiment relates to a method of performing automatic registration using a medical imaging system comprising automatically locating at least one sensor having at least one known identifier. A coordinate is then determined for the at least one sensor performing automatic registration. In at least one embodiment, such automatic registration may be used to generate a visual model.

One embodiment relates to a method of performing automatic registration using a medical imaging system comprising forming the at least one sensor having the known identifier. In one or more embodiments, the at least one known identifier may comprise at least one of a known artifact, a known shape and a geometrically known electromagnetic distorter. It is further contemplated the one method comprises fixing the at least one sensor to a patient.

Another embodiment relates to a method for performing automatic registration using a medical imaging system. This embodiment comprises automatically locating at least one known artifact in at least one sensor using an imaging system. A center of the at least one sensor is located using the imaging system and a coordinate is determined for the at least one sensor using an algorithm thereby generating a visual model.

Other embodiments of the method comprising embedding the at least one known artifact in the sensor, where the sensor may comprise a wireless sensor for example. The method may also comprise attaching the at least one sensor to a patient.

Still another embodiment relates to a method of performing automatic registration using a medical imaging system. This method may comprise automatically locating at least one sensor having a known shape using the imaging system. A coordinate of the sensor is determined using an algorithm for example, thereby generating a model.

One embodiment of the method may comprise forming the at least one sensor having the known shape. The at least one sensor may be fixed or connected to a patient.

Still another embodiment relates to a method of performing automatic registration using a medical imaging system. This embodiment comprises locating at least one cloud of sensor points using the imaging system. Coordinates for the at least one cloud of sensor points is determined using an algorithm for example.

One embodiment of the method comprising forming the at least one cloud of sensor points. The at least one cloud may be formed using at least one geometrically known electromagnetic distorter. The method further comprises attaching the at least one distorter to a patient.

Still another embodiment relates to a medical imaging system. This embodiment comprises a tracking module, an imaging module and a processing module. In at least one embodiment the tracking module is adapted to perform automatic registration of at least one sensor. The imaging module is adapted to locate an imaging space. The processing module communicates with at least one of the tracking and imaging modules, and is adapted to perform an automatic registration algorithm and generate a visual module.

In at least one embodiment of the imaging system, the tracking module is further adapted to locate the at least one sensor. The imaging system may further comprise a coupling device (a headset for example) communicating with at least one of the tracking, imaging and processing modules. Further, a display is contemplated communicating with at least the processing module and adapted to display the visual module, where a 3D visual module may be generated.

### BRIEF DESCRIPTION OF SEVERAL VIEWS OF THE DRAWINGS

FIG. 1 illustrates a block diagram of an embodiment of a medical imaging system, machine or device in accordance with certain embodiments of the present invention.

FIG. 2 illustrates a high level flow diagram depicting a method for performing automatic registration using an imaging system (similar to that illustrated in FIG. 1) in accordance with certain embodiments of the present invention.

FIG. 3 illustrates a flow diagram depicting a method for performing automatic registration using an imaging system (similar to that illustrated in FIG. 1) in accordance with certain embodiments of the present invention.

FIG. 4 illustrates a flow diagram depicting another method for performing automatic registration using an imaging system (similar to that illustrated in FIG. 1) in accordance with certain embodiments of the present invention.

FIG. 5 illustrates a flow diagram depicting a method for performing automatic registration using an imaging system (similar to that illustrated in FIG. 1) in accordance with certain embodiments of the present invention.

The foregoing summary, as well as the following detailed description of certain embodiments of the present invention, will be better understood when read in conjunction with the appended drawings. For the purpose of illustrating the invention, certain embodiments are shown in the drawings. It should be understood, however, that the present invention is not limited to the arrangements and instrumentality shown in the attached drawings.

### DETAILED DESCRIPTION OF THE INVENTION

For the purpose of illustration only, the following detailed description references a certain embodiment of a medical imaging system, machine, apparatus or device. However, it is understood that the present invention may be used with other medical devices or systems.

Techniques for reconstructing models (3D visual models for example) from tomographic two-dimensional images are known as provided previously. Some of these calibration techniques use markers which act as references in space during image acquisition (using for example an X-ray, ultrasound or other imaging device or system). The positions of these markers in three-dimensional space are supposed to be known. The image acquisition geometry for each projection may be deduced using equations which are derived from the position of the markers on the projected images.

In theory, techniques using markers should provide better precision than techniques that don't use markers. In practice, it is often difficult to precisely determine the position of the markers in space. It is contemplated that, the markers may move slightly or even be missed or acquired improperly by the system user during acquisition.

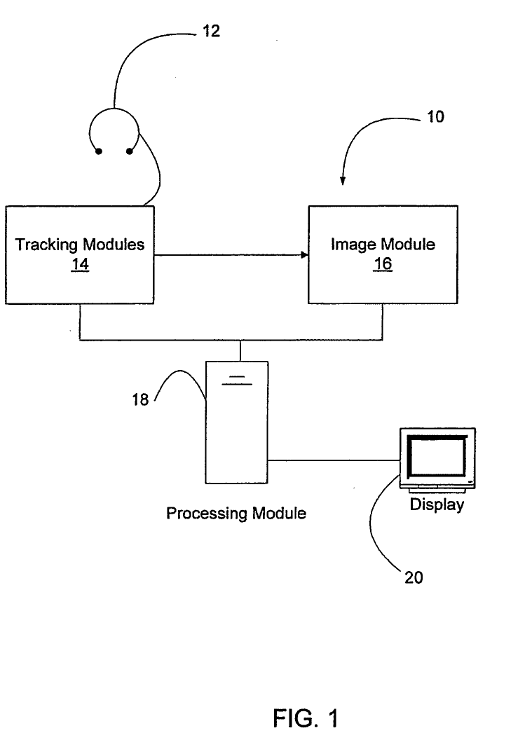

FIG. 1 illustrates one embodiment of a system, generally designated 10, adapted to perform fiducial registration in accordance with the embodiments of the present invention (automatically for example). One embodiment relates to a system adapted to perform fiducial based registration (wireless (disposable) and/or Electromagnetic (“EM”) distortion based fiducial based registration for example)).

In the illustrated embodiment, system 10 comprises at least one coupling module 12 having at least one sensor. In at least one embodiment, the sensor may comprise, for example, an electromagnetic-field receiver; and electromagnetic-field transmitter or an electromagnetic-field transponder (a receiver and transponder). In FIG. 1, coupling module 12 comprises a headset having a plurality of sensors arranged in a known pattern attached or couple thereto. Although a headset is depicted, any device or method for fixing or coupling one or more sensors to a patient's skin (or in a predetermined relationship thereto) is contemplated.

System 10 further comprises tracking and imaging modules 14 and 16, respectively. In FIG. 1, tracking and imaging modules 14 and 16 communicate with each other, and at least one of the modules 14, 16 communicate with coupling module 12. In one embodiment, at least tracking module 14 communicates with coupling module 12. Further, in the illustrated embodiment, both tracking and imaging modules 14 and 16 are depicted communicating with coupling module 12 using any suitable method (including wireless methods).

In at least one embodiment, the system 10 further comprises a processing module 18. In at least one embodiment, processing module 18 is adapted to perform one or more automatic registration algorithms to at least find the image coordinate. In at least one embodiment, the processing module 18 is adapted to compute one or more sensor coordinates without the surgeon or other user touching one or more of the sensors, and produce image coordinates using such automatic registration algorithms.

In at least one embodiment, the system 10 is further adapted to display one or more images, 3D visual models for example. In one embodiment, a display 20 is contemplated, wherein display 20 communicates with at least processing module 18. Display 20 may display panels such as, for example, MRI image and several x-ray fluoroscopic views.

One embodiment of system 10 is adapted to perform fiducial based registration (wireless (disposable) fiducial based registration for example). In at least one embodiment, a known artifact is embedded in a wireless sensor that may be wholly resolved using system 10. In at least one embodiment, multiple sensors may be attached to a patient prior to a scan using system 10.

The known artifact comprises the electrical center of the sensor. In at least one embodiment, the system 10 locates the artifact in the image space, and the tracking module 14 locates the center of the sensor. In this manner, the system 10 locates both matched pairs of the image and the sensor coordinates enabling a rigid transformation to be computed using processing module 18.

Alternatively, the sensor itself may be resolved with a known shape (a doughnut for example) may be found in the image space using system 10. In at least one embodiment the fiducial based registration is augmented by system computing the sensor coordinates. One embodiment of system 10 computes the sensor coordinates without the surgeon or other user actually touching any one of the markers. It is contemplated that the patient still needs to be scanned proximate the time of the surgery as the sensors need to be positioned in the same place during surgery as they were during the scan.

Still another embodiment uses geometrically known electromagnetic distorters in the sensor space forming a “cloud of points” for use in a surface registration. In at least one embodiment, 25 distorters would be used for a head using the current process. These distorters could play some part of a fixture that is affixed to the patient's heads so that they lie on, and in contact with, the patient's skin surface or at some predetermined relationship thereto (in a known vector offset from the skin for example).

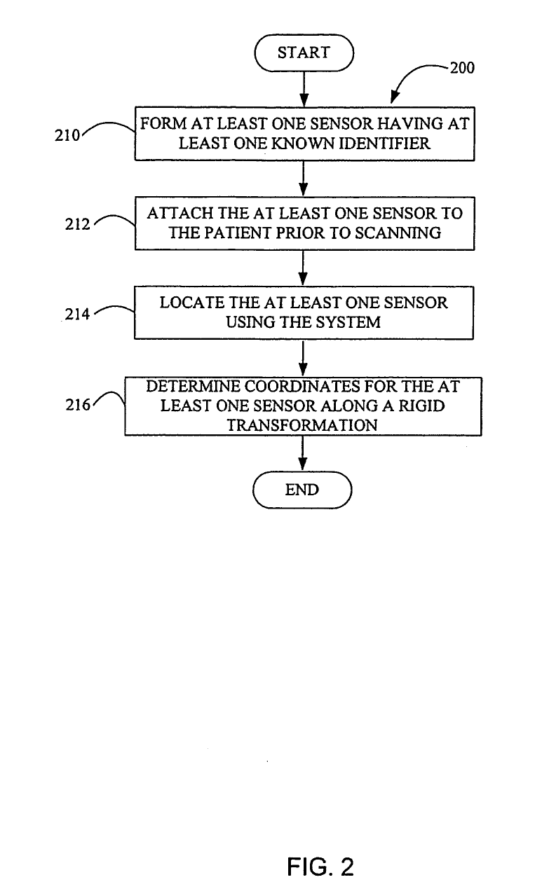

FIG. 2 illustrates a high level flow diagram depicting a method, generally designated 200, for performing automatic registration (fiducial based registration for example). In one embodiment, registration method 200 is performed using an imaging system or device (similar to that illustrated in FIG. 1).

In at least one embodiment, method 200 comprises Step 210, forming at least one sensor having at least one known or predetermined function or identifier (a known artifact, known shape or electromagnetic distortions for example). Method 200 further comprises Step 212, attaching the at least one sensor to a patient. In at least one embodiment, Step 212 comprises attaching multiple sensors to the patient (the patient's cranium for example) prior to a scan. Step 214 comprises locating at least one sensor. In at least one embodiment, multiple sensors are located using an imaging system. One embodiment of method 200 comprises Step 216, determining the coordinates for at least one sensor along rigid transformation using an algorithm for example.

In at least one embodiment, a known artifact is embedded in a wireless sensor that may be wholly resolved using a scanner for example. In at least one embodiment, multiple sensors having one or more known artifact may be attached to a patient prior to a scan.

It should be appreciated that, in this embodiment, the artifact is the electrical center of the sensor. In at least one embodiment, the system locates the artifact in the image space, and the tracking system locates the center of the sensor. In this manner, the system has located the both matched pairs of the image and the sensor coordinates enabling a rigid transformation to be computed.

Alternatively, the sensor itself may be resolved with a known shape the could be found in the image space. The basic idea behind at least one embodiment is that it is just another form fiducial based registration augmented by the idea that the tracking system computes the sensor coordinates. A proprietary method is known for finding image coordinates via AFFA. One embodiment computes sensor coordinates without the surgeon actually touching any markers. The major practical drawback to this idea is that the patient still needs to be scanned approximate the time of the surgery as the sensors need to be in the same place during surgery as they were during the scan.

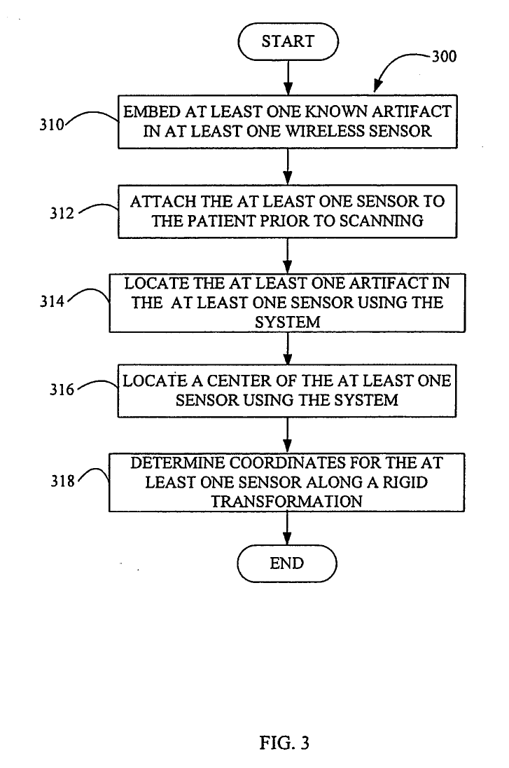

FIG. 3 illustrates a flow diagram depicting a method, generally designated 300, for performing automatic registration (wireless or disposable fiducial based registration for example) in accordance with at least one embodiment of the invention. In one embodiment, registration method 300 uses an imaging system or device (similar to that illustrated in FIG. 1).

One embodiment of method 300 comprise Step 310, embedding at least one known artifact in at least one sensor (a wireless sensor for example). In at least one embodiment, one or more artifacts may be embodied in one or more sensors prior to the sensors being attached to a patient. Further, in one embodiment, at least one artifact is formed as the electrical center of the at least one sensor.

Step 314 comprises locating the at least one artifact. In at least one embodiment, the imaging system locates the artifact in the image space, and the tracking system locates the center of the sensor as illustrated by Step 316. In this manner, the system located both matched pairs of the image and the sensor coordinates enabling a rigid transformation to be computed. Step 318 comprises determining to the coordinates along a rigid transformation using an algorithm for example.

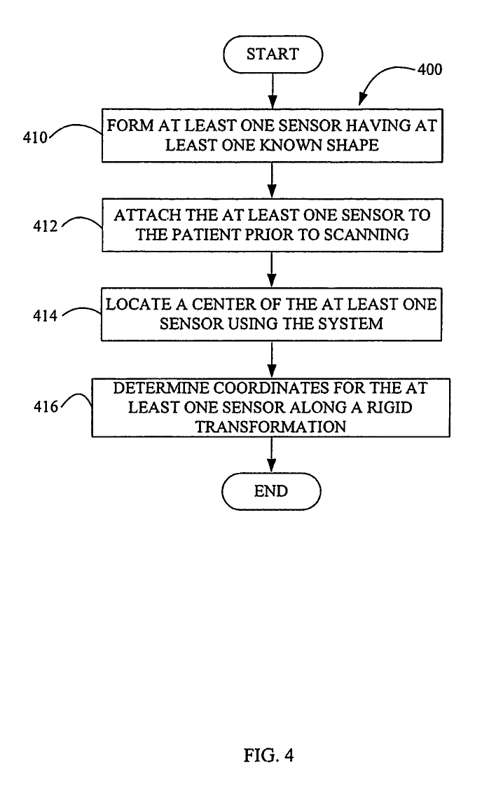

FIG. 4 illustrates a flow diagram depicting a method, generally designated 400, for performing automatic wireless registration (wireless fiducial based registration) using an imaging system or device similar to that illustrated in FIG. 1. In the illustrated embodiment, method 400 comprises Step 410, forming at least one sensor having at least one known shape. Method further comprises Step 412, attaching the at least one sensor to the patient prior to scanning. In at least one embodiment, the at least one or more sensors are attached to the patient using a coupling module or headset as illustrated previously.

Method 400 further comprises Step 414, locating a center of the at least one sensor using the system. Method 400 further comprises Step 416, determining coordinates for at least one sensor along a rigid transformation.

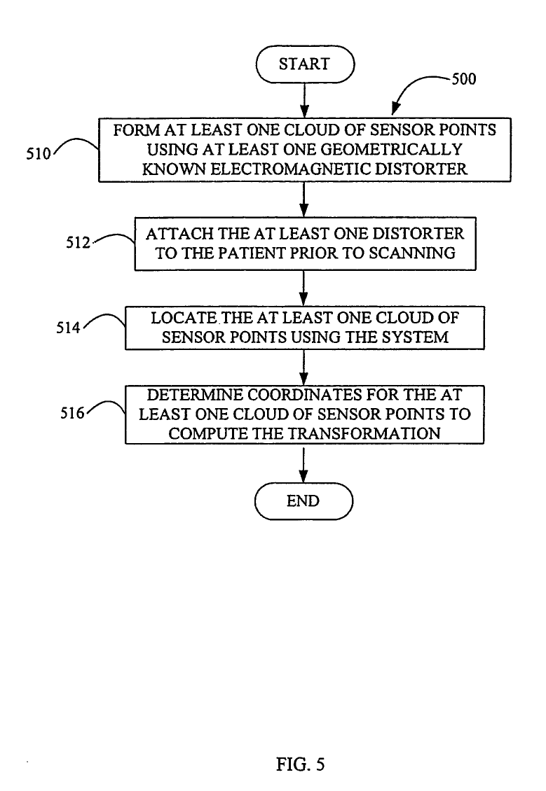

FIG. 5 illustrates a flow diagram depicting a method for performing automatic registration (electromagnetic based registration) using an imaging system or device similar to that depicted previously. In the illustrated embodiment, method 500 comprises Step 510 forming a cloud of sensor points. In at least one embodiment, the at least one cloud of sensor points is formed using at least one geometrically known electric magnetic distorter. Method 500 further comprises Step 512, attaching the at least one distorter to the patient prior to scanning.

In at least one embodiment, method 500 comprises Step 514 locating the at least one cloud of sensor points using the system. Step 516 comprises determining coordinates for the at least one cloud of sensor points to compute the transformation.

While the invention has been described with reference to certain embodiments, it will be understood by those skilled in the art that various changes may be made and equivalents may be substituted without departing from the scope of the invention. In addition, many modifications may be made to adapt a particular situation or material to the teachings of the invention without departing from its scope. Therefore, it is intended that the invention not be limited to the particular embodiment disclosed, but that the invention will include all embodiments falling within the scope of the appended claims.

1. A method of performing automatic registration using a medical imaging system comprising:
automatically locating at least one sensor having at least one known identifier; and
determining a coordinate for said at least one sensor and performing automatic registration.

2. The method of claim 1 comprising forming said at least one sensor having said known identifier.

3. The method of claim 1 comprising fixing said at least one sensor to a patient.

4. The method of claim 1 wherein said at least one known identifier comprises at least one of a known artifact, a known shape and a geometrically known electromagnetic distorter.

5. A method for performing automatic registration using a medical imaging system comprising:
automatically locating at least one known artifact in at least one sensor using an imaging system;
locating a center of said at least one sensor using said imaging system; and
determining a coordinate for said at least one sensor using an algorithm thereby generating a visual model.

6. The method of claim 5 comprising embedding said at least one known artifact in said sensor.

7. The method of claim 5 wherein said sensor comprises a wireless sensor.

8. The method of claim 5 comprising attaching said at least one sensor to a patient.

9. A method of performing automatic registration using a medical imaging system comprising:
automatically locating at least one sensor having a known shape using the imaging system; and
determining a coordinate of said sensor using an algorithm, thereby generating a visual model.

10. The method of claim 9 comprising forming said at least one sensor having said known shape.

11. The method of claim 9 comprising fixing said sensor to a patient.

12. A method of performing automatic registration using an imaging system comprising:
locating at least one cloud of sensor points using said imaging system; and
determining coordinates for said at least one cloud of sensor points using an algorithm.

13. The method of claim 12 comprising forming said at least one cloud of sensor points.

14. The method of claim 13 comprising forming said at least one cloud using at least one geometrically known electromagnetic distorter.

15. The method of claim 14 comprising attaching said at least one electromagnetic distorter to a patient.

16. A medical imaging system comprising:
a tracking module adapted to perform automatic registration of at least one sensor;
an imaging module adapted to locate an imaging space; and
a processing module communicating with at least one of said tracking and imaging modules, adapted to perform an automatic registration algorithm and generate a visual module.

17. The imaging system of claim 16 wherein said tracking module is further adapted to locate said at least one sensor.

18. The imaging system of claim 16 comprising a coupling module communicating with at least one of said tracking, imaging and processing modules.

19. The imaging system of claim 16 comprising a display communicating with at least said processing module and adapted to display said visual module.

20. The imaging system of claim 16 wherein said processing module generates a 3D visual module.
